(19) United States
(12) Patent Application Publication
(10) Pub. No.: US 2020/0277650 A1
(43) Pub. Date: Sep. 3, 2020

(54) POLYNUCLEOTIDE-BINDING PROTEIN FOR USE IN DIAGNOSIS

(71) Applicant: Matjaz Vogelsang, Celje (SI)

(72) Inventor: Matjaz Vogelsang, Celje (SI)

(21) Appl. No.: 15/733,101

(22) PCT Filed: Nov. 13, 2018
(86) PCT No.: PCT/IB2018/058933

§ 371 Date: May 16, 2020

Related U.S. Application Data
(60) Provisional application No. 62/587,003, filed on Nov. 16, 2017.

(51) Int. Cl. C12Q 1/6806; G01N 33/493

(52) U.S. Cl. CPC C12Q 1/6806; G01N 33/493; C12Q 2522/101; G01N 2800/26; G01N 2800/7028; G01N 2800/24; G01N 2800/32; G01N 2333/245

(57) ABSTRACT

The present disclosure relates to a polynucleotide-binding protein for use in in vitro diagnostics. The present disclosure also relates to a method of separation of nucleic acids from a liquid biological sample, protecting nucleic acids in a biological sample from degradation, a method of extracting nucleic acids from a solution and a kit or container for protecting and/or separating nucleic acids from a solution, and more specifically to a method for preserving and/or separating nucleic acids from a sample.

### CROSS-REFERENCE TO RELATED APPLICATION

This application claims priority to, and the benefit of, U.S. Provisional Application No. 62/587,003, filed Nov. 16, 2017, the content of which is hereby incorporated by reference in its entirety.

### SEQUENCE LISTING

The instant application contains a Sequence Listing which has been submitted electronically in ASCII format and is hereby incorporated by reference in its entirety. Said ASCII copy, created on Nov. 4, 2018, is named PCT001_2018_ST25.txt and is 2.526 bytes in size.

### FIELD OF THE DISCLOSURE

The present disclosure relates in general to a field of biomedical laboratory science, more specifically to the field of diagnostic, specifically to liquid biopsy. The present disclosure relates to a method of separation of nucleic acids from a liquid biological sample, protecting nucleic acids in a solution from degradation, a method of extracting nucleic acids from a solution and a kit or container for protecting and/or separating nucleic acids from a solution, and more specifically to a method for preserving and/or separating nucleic acids from a sample. More specifically the present disclosure relates to methods, kits and means for separating circulating cell-free (cfNAs) from a sample, which in turn allows first optionally further purification, and then processing or analysis of the purified nucleic acid.

### BACKGROUND OF THE DISCLOSURE

Liquid biopsy is the analysis of molecular biomarkers in a liquid sample obtained from a subject containing for example circulating cell-free DNA (ccfDNA or cfDNA), circulating cell-free RNA (cfRNA), micro RNA (miRNA), circulating tumor DNA (ctDNA), cellular DNA, cellular RNA, circulating tumor cells (CTCs) and extracellular micro-vesicles (such as exosomes) containing RNA, proteins, and lipids. Biomarkers, such as an increased copy number, mutation, translocation or the like, can be detected in the sample. Generally, the biomarkers are very condition-specific. The presence of a biomarker characteristic of a physiological process, pathological process or a disease can thus inform the physician of a treatment options. The fields where liquid biopsy is mainly performed are oncology and prenatal testing, but the method holds value in other biomedical areas, including transplantation medicine, cardiovascular care, traumatology, autoimmune disease, virology and microbial infections. An April 2017 review article in Nature Reviews, Vol 17, 223-38 reports on possible applications of liquid biopsies based on ctDNA. The benefit of the liquid biopsy is that it is non-invasive or much less invasive compared to the solid biopsy. However, the broad use of liquid biopsy is currently hindered by low sensitivity of the methods. The methods do not enable to isolate enough quality analyte from a biological sample to allow robust detection. The reasons are mainly low isolation yield of cell-free nucleic acids (cfNA) from a biological sample, short half-life of cfNA and degradation of cfNA during and after sample collection, storage and transport. cfDNA was first reported by Lo, Y. M. D. et al. in Presence of fetal DNA in maternal plasma and serum published in Lancet (1997), 350, 485-487. Still, even after about 20 years robust methods for separation and isolation of cfDNA from biological samples are lacking. In WO2011/083429 DNA isolation methods from solid or liquid sample are described that use cotton. Application claiming benefit of U.S. Ser. No. 60/269,729, filed on Feb. 16, 2001, discloses several other nucleic acid isolation methods in the background section together with the methods utilizing magnetizable cellulose or magnetizable cellulose derivatives, in presence of certain concentrations of salt and polyalkylene glycol to bind nucleic acids.

Nevertheless, improved methods of isolating or separating nucleic acid from the biological sample are needed in order to aid liquid biopsy diagnostic test development.

### SUMMARY OF THE DISCLOSURE

The present invention provides, inter alia, a solution, which makes it possible to 1) preserve polynucleotides in a biological sample obtained from a subject; 2) to separate polynucleotides from a biological sample and 3) whilst it works in smaller volumes as well, to accumulate polynucleotide even from a larger liquid-sample volume, such as volumes above 4 ml, above 10 ml, 50 ml or more, for example 500 ml, 1000 ml, or the like, which yields sufficient polynucleotides for a sufficiently sensitive liquid biopsy-related analysis or diagnostics.

Surprisingly, it was found that polynucleotides can be effectively separated from a biological sample by employing a polynucleotide-binding protein. By applying a polynucleotide-binding protein, polynucleotides can be separated from the sample conveniently by a simple centrifugation, filtration or other separation methods. In addition, when bound to the polynucleotide-binding protein, polynucleotides in a sample remain stable even during handling and/or transport. The polynucleotide-binding protein forms a complex with a polynucleotide and the complex sustains a wide range of stresses, including oxidative stress, high pressure, UV and gamma radiation, pH shock, iron and copper toxicity and high temperatures (e.g. up to 100° C.). Furthermore, the use of polynucleotide-binding protein can be easily scaled-up in terms of a sample volume from which polynucleotides are separated. The reason is that the preparation of a polynucleotide-binding protein is an uncomplicated and straightforward process, which makes large quantities of the protein accessible in a timely and low-cost manner. As a reagent that binds to polynucleotides in a sample, a polynucleotide-binding protein can thus be easily used in sufficient quantities, even in large volumes. The above aspects present a concrete advantage of the instant disclosure over the existing methods for DNA/RNA separation that are based on magnetic beads-technology. Magnetic beads are more expensive to produce, which consequently also limits their use in larger sample volumes; and magnetic beads do not provide comparable protection to the isolated polynucleotides.

It was shown herein that a polynucleotide-binding protein provides suitable means to efficiently separate polynucleotides from a biological sample. This was shown also for samples of about 50 ml, which just exemplifies how broad the present invention can be applied—for example in terms of the biological sample (e.g. urine sample), and offers a solution to increase the sensitivity of liquid biopsy methods by enabling capturing more analyte by accumulating polynucleotides from larger volumes.

Particularly, a suitable polynucleotide-binding protein used can be a DNA-binding protein from starved cells, also termed Dps protein, or by analogy a Dps-like protein. However, the present disclosure based on a Dps protein sets a principle for any polynucleotide-binding protein that forms a complex with a polynucleotide (e.g. nucleoid-associated proteins) in a solution and the complex precipitates.

In one aspect, the present disclosure provides a method comprising directly contacting a polynucleotide in a liquid biological sample from a subject in vitro with a polynucleotide-binding protein, thereby forming a protein-polynucleotide complex that precipitates in the sample.

In another aspect, the present disclosure provides use of a polynucleotide-binding protein in a method for separation of polynucleotides from a liquid biological sample from a subject.

In yet another aspect, the present disclosure provides use of a polynucleotide-binding protein in a method for protecting a polynucleotide in a biological sample from a subject.

In further aspect, the present disclosure provides a method for protecting a polynucleotide in a liquid biological sample comprising polynucleotides from a subject comprising a step of adding a polynucleotide-binding protein to the biological sample.

In another aspect, the present disclosure provides a method to bind polynucleotides in a liquid biological sample from a subject to a polynucleotide-binding protein, the method comprising adding the polynucleotide-binding protein to the liquid biological sample comprising polynucleotides.

In the sixth aspect, the present disclosure provides a method for separation of polynucleotides from a liquid biological sample comprising polynucleotides from a subject, or a lysate thereof, the method comprising the steps of:
(a) providing the liquid biological sample obtained from a subject, or the lysate thereof, comprising polynucleotides;
(b) adding a polynucleotide-binding protein to the sample, or the lysate thereof, to form a protein-polynucleotide complex; and
(b) separating the formed protein-polynucleotide complex from the sample, or from the lysate thereof.

In another aspect, the present disclosure provides a kit for use in the method comprising directly contacting a polynucleotide in a liquid biological sample from a subject in vitro with a polynucleotide-binding protein, thereby forming a protein-polynucleotide complex that precipitates in the sample.

In yet another aspect, the present disclosure provides a container for collection of a biological sample from a subject comprising a polynucleotide-binding protein.

In a further aspect, the present disclosure provides an isolated protein-polynucleotide complex comprising a polynucleotide-binding protein and a polynucleotide from a subject.

Further aspects provided by the present disclosure are described in the claims 88, 89 and 90. Another aspect of the present disclosure is use of polynucleotide-binding protein in the in vitro diagnosis of a disease or a condition of a subject, wherein the polynucleotide-binding protein is used to separate a polynucleotide from a liquid biological sample from the subject comprising the polynucleotide.

Further aspect of the present disclosure is a method for detecting the presence of a biomarker in a biological sample from a subject, said method comprising:

(i) providing the liquid biological sample obtained from a subject comprising polynucleotides;

(ii) adding a polynucleotide-binding protein to the sample to form a protein-polynucleotide complex;

(iii) separating the formed protein-polynucleotide complex from the sample; and

(iv) assaying the polynucleotide to determine whether the biomarker is present in the liquid biological sample from said subject.

In another aspect the present disclosure provides a method of characterizing a disease or a condition of a subject comprising detecting the presence of a biomarker indicative of said disease or the condition in a liquid biological sample from the subject, the method comprising:

(i) providing the liquid biological sample obtained from a subject comprising polynucleotides;

(ii) adding a polynucleotide-binding protein to the sample to form a protein-polynucleotide complex;

(iii) separating the formed protein-polynucleotide complex from the sample; and

(iv) assaying the polynucleotide to determine whether the biomarker is present in the liquid biological sample from said subject.

### BRIEF DESCRIPTION OF THE DRAWINGS

FIG. 1: Various amounts of DNA can be separated from 1 mL solution using Dps protein (10 μL). DNA yields before (empty bars) and after (black bars) separation are comparable, suggesting complete recovery of DNA from a solution. Error bars represent 5% coefficient of variation (CV) of the fluorescence measurement.

FIG. 2: Concentrating DNA from a large volume solution (50 mL) containing DNA. Amount of DNA was measured fluorometrically in the starting solution (empty bars), in supernatant decanted after centrifugation (striped pattern) and in the final resuspension of the pellet (black bars). DNA detection limit was below 5 pg/μL.

FIG. 3: RNA can be separated from a solution using Dps protein. Amount of RNA was measured fluorometrically in the starting solution (empty bars), in supernatant decanted after centrifugation (striped pattern) and in the final resuspension of the pellet (black bars). RNA detection limit was below 0.2 ng/μL.

FIG. 4: Collection of spiked-in DNA from urine sample using Dps protein. Amount of DNA was measured fluorometrically in the starting solution (empty bars), in supernatant decanted after centrifugation (striped pattern) and in the final resuspension of the pellet (black bars). DNA detection limit was below 5 pg/μL. DNA amount is presented (y-axis) in proportion (%) to DNA amount in the starting solution of each respective sample.

FIG. 5: Separation of cfDNA from urine using Dps protein. Amounts of DNA extracted from 16 mL of cell-free urine sample from one healthy individual with different amount of added Dps are shown. DNA was measured fluorometrically.

FIG. 6: Dps protein protects DNA from DNaseI-mediated cleavage. Dps protein was able to shield DNA from DNase I cleavage for at least 9 days in water. The figure shows A) Input DNA analysed by Bioanalyzer 2100 (Agilent Technologies); B) analysed DNA in the water sample with DNase I and Dps protein after 9 days at room temperature; C) analysed DNA after 9 days at room temperature in the water sample with DNase I, but without Dps protein.

FIG. 7: Dps protein was able to stabilize DNA and to shield it from degradation for at least 9 days in a liquid biological sample (biofluid), such as urine. The figure shows A) Input DNA analysed by Bioanalyzer 2100 (Agilent Technologies); B) analysed DNA in the urine sample with Dps protein added after 9-day storage at room temperature; C) analysed DNA in the urine sample without Dps protein protection after 9-day storage at room temperature.

FIG. 8: High yield of pure cfDNA obtained from larger volume by purifying cfDNA with both, Dps protein and with the QIAamp MinElute ccfDNA Mini kit (QIAGEN) (upper curve), compared to the lower amount of cfDNA obtained by purifying it solely with the QIAamp MinElute ccfDNA Mini kit (QIAGEN) (bottom curve). Adding a process step of purifying nucleic acids from a biological sample by using Dps protein to a purification method known in the art allows to obtain nucleic acids from volumes larger than those used in the respective purification methods known in the art. In addition, the obtained nucleic acid is of better quality.

### DETAILED DESCRIPTION OF THE DISCLOSURE

The finding that polynucleotide-binding proteins can be used to bind polynucleotides in a biological sample led to the use of polynucleotide-binding proteins in in vitro diagnostics. An exemplary method employing the principle comprises directly contacting a polynucleotide in a liquid biological sample from a subject in vitro with a polynucleotide-binding protein, thereby forming a protein-polynucleotide complex that precipitates. While being inert to a biological sample, polynucleotide-binding proteins can be used in sufficient quantities to capture and isolate substantial amount of polynucleotides from a biological sample, even if the sample is of a sizeable volume. Customarily, cell free nucleic acids have been isolated from samples of up to 10 ml, which would equate to a volume of a large blood collection tube, and often much less than that. The expensive magnetic-beads technology is not meant to handle sample volumes larger than 2 ml. Contrary to that, the present disclosure provides means to advantageously collect nucleic acids, including cell-free nucleic acids, from practically any volume. The limiting factor then becomes the size of a sample container and possible inconvenience of handling large volumes. However, the sample volume can be reduced by splitting the large-volume sample to several aliquots, while each of them can still be for example 500 ml (e.g. collection of urine over the whole day). The benefit of collecting larger sample volumes lies in a possibility to catch sufficient number of polynucleotides in general; including those that are expectedly present only in scarce numbers. For example, a small non-metastasized early stage cancer is not expected to produce large amounts of ctDNA that would allow easy detection. Therefore, applying polynucleotide-binding proteins to adequately scavenge large sample volumes, makes polynucleotide-binding proteins particularly suitable for use in in vitro diagnostics (e.g. methods conducted for example in a biomedical laboratory that are separated, distinct from and do not include methods or steps practised on a living subject, such as for example to collect a biological sample from said subject). In one embodiment, the methods, processes, uses as disclosed herein are used in vitro.

The term “polynucleotide-binding protein” as used herein denotes a protein or a part thereof that can as a monomer, oligomer or a polymer together with a polynucleotide in water form a complex and the formed complex precipitates. Preferably, a polynucleotide-binding protein denotes a protein or a part thereof that, as a monomer, oligomer or a polymer forms a complex with a polynucleotide in water that precipitates at pH 7 when the concentration of the polynucleotide in water is at least 12 ng/mL and the concentration of the polynucleotide-binding protein is at least 70 μg/μL, 7 μg/μL. preferably precipitates already at the polynucleotide-binding protein concentration of at least 70 pg/μL. The polynucleotide-binding protein is not a clinical analyte nor an antibody. The solubility of the formed complex is such that it precipitates from the sample solution. The solubility of the complex may be further dependent on the concentration of divalent metal ions, but the basic test for a suitability of a protein or a part thereof to be applied as a polynucleotide-binding protein is conducted in water devoid of such metal ions. The polynucleotide-binding protein can be for example a nucleoid-associated protein. As described by Shane C. Dillon and Charles J. Dorman in Nature Reviews Microbiology 8, 185-195 (March 2010) a nucleoid-associated proteins possess DNA-binding activity and an ability to alter the trajectory of the DNA molecule (that is, the direction taken by the DNA through three-dimensional space) by bending, wrapping or bridging it. The proteins can be prepared in prokaryotic as well as eukaryotic expression systems. In one embodiment, the polynucleotide-binding protein is a prokaryotic protein. The formation of the complex can be measured by gel-shift electrophoresis and the precipitation of the complex can be measured spectroscopically: for example by dynamic light scattering (DLS) technique or by turbidity measurement. The preferred polynucleotide-binding protein is a bacterial protein. The polynucleotide-binding protein can comprise also a mixture of more than one protein other than an antibody that binds a polynucleotide in a solution to form a complex and the formed complex precipitates. The most preferred polynucleotide-binding protein is a Dps protein or a Dps-like protein. In one embodiment, the polynucleotide-binding protein is a Dps protein.

Variants of the aforementioned polynucleotide-binding protein are also contemplated herein. For example, a polynucleotide-binding protein having one or more amino acids in its sequence changed, replaced, deleted or added, or a part thereof, that can still bind a polynucleotide in a solution to form a complex that precipitates, is adequate to still perform its function in the methods described herein. A polynucleotide-binding protein can also be fused to another peptide or protein, either directly or via linker. Recombinant DNA techniques available to modify proteins by substitution, insertion or deletion of one or more amino acids are well known in the art. A possible modification to proteins such as Dps proteins would be to eliminate the iron binding activity by changing the amino acids responsible for the binding of iron while retaining the nucleic acid binding activity.

The term “Dps protein” as used herein refers to a DNA-binding protein from starved cells (Dps) that forms a complex with a polynucleotide as described above. Dps proteins were first characterized by Almirón M. et al. in A novel DNA-binding protein with regulatory and protective roles in starved Escherichia coli in Genes Dev. (1992) December; 6(12B):2646-54. Dps are proteins found in bacteria (97%) and archaea (3%), but not in animals or humans. Dps proteins are often composed of 12 identical monomers (with molecular mass of about 19 kDa each) assembled into a hollow, tetrahedrally symmetric sphere with a diameter of about 9 nm and an inner cavity of about 4.5 nm in diameter. While the dodecameric nanoparticle structure is well conserved among all Dps, the Dps primary amino acid sequences vary widely among different families of bacteria or archaea, with some sharing less than 30% amino acid sequence identity. Certain examples of Dps proteins are Dps of E. Coli (Swiss-Prot Accession # P0ABT2), Mycobacterium smegmatis (Swiss-Prot Accession # P00558), Lactobacillus, Bacteroides, extremophilic or hyperthermophilic bacteria or archaea such as Sulfolobus solfataricus or Deinococcus radiodurans. In one embodiment, the Dps protein is the protein known under the accession number NP_415333.1.

Dps protein can interact with both supercoiled and linear nucleic acid to form the complex, namely a dense biocrystal structure, resulting in physical protection of nucleic acids against wide range of stresses, including oxidative stress, high pressure, UV and gamma radiation, pH shock, iron and copper toxicity and high temperatures (i.e. up to 100° C.). In addition to polynucleotide binding, Dps protein can exhibit other properties, such as metal binding and sequestration, ferroxidase activity and ability to affect gene regulation. Dps proteins are mini-ferritins with many structural and functional similarities to maxi-ferritins (Ftn and Bfr proteins) and together they form the ferritin superfamily of iron storage proteins with magnetic properties. It is known that Dps binds to polynucleotides without apparent sequence specificity, forming a highly stable complex and compacting the polynucleotides into a highly ordered crystalline structure (i.e. biocrystals), however, the exact binding mechanism is still not completely understood. The highly organized co-crystallization process is obtained with supercoiled plasmids, linear double-stranded DNA, single-stranded DNA, as well as single-stranded RNA molecules without a significant difference in the resultant crystalline structure. The affinity of Dps for polynucleotides is sensitive to buffer conditions. Dps protein binds polynucleotides weaker in the presence of higher salt concentrations. For example, the formation of a complex, i.e. biocrystals, can depend on the concentration of divalent cations such as Mg2+, where a concentration of up to 1 mM Mg2+ supports the assembly of protein-polynucleotide complexes, while the concentration of more than about 3 mM Mg2+ disrupts the formation of the complex. The Dps proteins can be for example those that are found in bacteria strains of Streptococci, Listeria, Helicobacter and Escherichia. Preferably the Dps protein is from Escherichia Coli.

The term “Dps-like protein” refers to any recombinant or isolated natural protein or a variant thereof other than a Dps protein that exhibits functional similarity with a Dps protein of forming a precipitating complex with a polynucleotide as described above. Pyrococcus furiosus proteins encoded by the PfDps gene are known to be Dps-like proteins (Ramsay, B. et., J Inorganic Biochem., 100 (2006) 1061-1068.

The term “polynucleotide” as used herein refers to DNA, RNA or polyamide nucleic acid. The term means DNA (single- or double-stranded) or RNA. The polynucleotide can include genomic DNA, cell-free nucleic acids (cfNA), circulating cell-free DNA (cfDNA), circulating cell-free RNA (cfRNA), micro RNA (miRNA), circulating tumor DNA (ctDNA), circulating tumor RNA (ctRNA), peptide nucleic acid, polyamide nucleic acid and nucleic acids in extracellular micro-vesicles (such as exosomes containing RNA, proteins, and lipids). Preferably, the polynucleotide is cell-free nucleic acids (cfNA). In oncology, the most preferred polynucleotide is circulating tumor DNA (ctDNA). In one embodiment, polynucleotide that can be bound by polynucleotide-binding protein can be up to 1000 base pairs (bp) long. Preferably, the polynucleotide contains between 50 and 200 bp, specifically between 50 and 166 bp, or between 134 and 144 bp.

As used herein, the meaning of an “a” or of a singular noun refers also to plural and includes that of a plural noun. A singular term, unless otherwise indicated, can also carry the meaning of its plural form.

One embodiment of the present invention is to apply polynucleotide-binding protein in diagnostics in a manner to help extract polynucleotides from a liquid biological sample. Such use of the polynucleotide-binding protein in separation and detection methods can avail physicians to obtain crucial information about the condition or a disease of a subject in a more reliable manner. Adding the protein to the sample obtained from a subject causes the protein to form a complex with the present free-floating (cell-free) polynucleotides, which in turn precipitates. The polynucleotide-binding protein can capture only polynucleotides outside a cell. One possibility is to first disrupt the cells to release and thus expose nucleic acids to the binding of the polynucleotide-binding protein. A polynucleotide-binding protein can thus be uses to separate a polynucleotide from a biological sample. The term “biological sample” as used herein refers to a biological specimen from a subject including, for example plasma, serum, blood, sputum, tear, urine, saliva, synovial fluid, pleural effusions or cerebrospinal fluid, cell, tissue, stool. In the preferred embodiment, the biological sample refers to a liquid biological specimen from a subject, such as plasma, serum, blood, sputum, tear, urine, synovial fluid, saliva, pleural effusions or cerebrospinal fluid, or a lysate thereof. The sample can be also initially solid, such as obtained from a solid biopsy, but converted into a liquid sample, preferably already in a presence of a polynucleotide-binding protein. For example, cells, tissue from a solid biopsy or paraffin embedded tissue block, or a slice thereof can be liquified by suspending in water (preferably nuclease-free water) by lysis or other technique known to a skilled person and thus a liquid biological sample prone to benefit from the methods of the present disclosure can be prepared. Preferably, the biological sample is plasma, serum or urine, most preferably is plasma. The sample is obtained from a subject. The term “subject” as used herein refers to a mammal. Mammal can be healthy or is suffering from a disease or is having a condition. The disease is one that can be diagnosed directly or indirectly by analysis of a biological sample from the subject. An example of the disease is cancer. Another example is infection. A condition can be for example pregnancy, or carrying a transplant organ. In the preferred embodiment the subject is human. In one embodiment the subject is a cancer patient. In an alternative embodiment the subject is a patient with infection. In yet another embodiment, the subject is a transplant organ recipient.

While the polynucleotide-binding protein binds to polynucleotides in a sample it also protects them from stressors. The polynucleotide-binding protein in a complex, i.e. biocrystal offers polynucleotide physical protection. Among others, this makes samples insensitive to temperature fluctuations during transport and storage. As described above, the proteins are envisioned to stabilize polynucleotides in a solution in multiple ways. The terms “protect” or “stabilize” can be used interchangeably and as used herein in relation to a polynucleotide mean preventing or reducing degradation or cleavage of the polynucleotide. The term includes protection of the polynucleotide against a wide range of stresses, for example against nucleases, oxidative stress (such as the one caused by hydroxyl radicals or the presence of iron or copper ions), high pressure, ultraviolet light (UV light), gamma radiation, a change in pH for more than 5 units on the pH scale, and/or high temperature (i.e. up to 100° C.). Preferably, to protect or to stabilize means herein to reduce degradation of a polynucleotide by nuclease or oxidative stress, preferably the terms mean to reduce degradation of a polynucleotide by nuclease. The result of protecting or stabilizing is preservation of polynucleotides in a sample. The polynucleotides in a sample retain their original length and their initial sequence. Keeping the polynucleotides in a sample protected or stabilized, i.e. intact, allows for a more reliable, detailed and robust analysis and consequently better diagnosis. We have observed that a Dps protein can satisfactorily stabilize or protect nucleic acids in the initial biological sample, or in the presence of nucleases, for at least 9 days at room temperature (25° C.). It can be easily conceived that the period of effective protection achieved by a polynucleotide-binding protein is much longer if the complex of a polynucleotide-binding protein and polynucleotide is removed from the biological sample, and further if the complex is purified.

The protective feature of a polynucleotide-binding protein can be beneficially exploited in liquid biopsy settings. The low sensitivity of the current methods is caused in part of the fact that nucleic acids, particularly those outside of cells, are prone to be degraded by nucleases. The degradation continues during the sample collection, handling and storage. The solution of the present disclosure addresses this issue by protecting the present cell-free nucleic acids possibly as soon as the sample is obtained. This feature of the present disclosure can also be valuably applied to stabilize the samples in situations where extended transport times for a biological sample are expected or where cooling down or freezing are not readily available. For example, the polynucleotide-binding protein can be efficiently applied to stabilize the sample comprising polynucleotides, e.g. for later down-stream processing and analysis, in remote areas, areas with disrupted electricity supply, developing countries, or the like.

The disclosure can be conveniently put into practice by adding the polynucleotide-binding protein to a liquid sample comprising the free-floating polynucleotides to form a protein-polynucleotide complex, and the protein-polynucleotide complex is separated from the liquid biological sample. Once added to the sample, polynucleotide-binding protein precipitates cell-free nucleotides comprised in the sample. The binding of the protein to the polynucleotides in the sample and thus forming of the complex protein-polynucleotide can be improved by incubating the sample at lower temperatures. For example, the incubation of the sample at 4° C. improves the yield of finally recovered or isolated polynucleotides from the sample. The precipitation yield can be dependent on the pH of the sample and concentration of divalent-metal ions. The polynucleotide-binding protein forms the complex with nucleic acids in a wide pH range. Nucleic acids get precipitated at least at pH less than 7, but also higher pH can work; for example between pH 3 and pH 8. It can be also observed in literature that a Dps protein can bind to DNA for example at low pH of 2.2 or pH 3.6. The concentration of ions can be reduced by adding a chelating agent such as EDTA, or increased by adding them to the sample. The concentration of divalent metal ions needed for optimal binding of a respective polynucleotide-binding protein can be determined by preparing series of standard water solutions comprising polynucleotides, such as a standard ladder of known size and concentration, and increased concentrations of the metal ions. The result of such optimization experiment can also help to define what change in divalent metal ion concentration is required for the polynucleotide-binding protein to release the polynucleotide from a complex, for example after separating the complex from the sample. Generally, once the complex of the polynucleotide-binding protein and polynucleotides from a sample solution precipitates, the precipitated complex can be separated from the sample by any known method to separate the precipitate. For an example, the protein-polynucleotide complex can be separated by chromatography (e.g. using Sephadex G-15 column), centrifugation, sedimentation, filtration of the sample, use of magnetic particle technologies, or by utilizing magnetic properties of the polynucleotide-binding protein. Another option to separate assembled complex from the sample when a Dps protein is used is by utilizing magnetic properties of the Dps protein. Dps proteins have the ability to oxidize Fe2+ to paramagnetic Fe3+, which is deposited inside a protein cavity. This results in the formation of an iron core with magnetic properties, which can be exploited for separation of polynucleotide-Dps protein complexes. Preferably, the complex is separated by centrifugation. Centrifugation is deemed as one of the basic laboratory technique. This feature of the disclosure thus corroborates further advantages of the present disclosure, which are effortless use and broad applicability of the disclosure.

Once a protein-polynucleotide complex is separated from the sample, it can be stored for later use (preferably by freezing it), purified, or broken up to release nucleic acids from the complex. These actions can be performed in any order and the final protocol can be optimized. Such downstream handling of the separated complex can be combined with any suitable separation, purification or analysis method, including purification with magnetic particle technology. Release of the polynucleotide from the complex can be achieved by hydrolysis of the polynucleotide-binding protein, for example by proteinase, such as proteinase A or K. When the polynucleotide-binding protein is a Dps protein, the nucleic acid can be released by resuspending the complex in a solution comprising generally increased concentration of Mg2+ ions. Increased molar concentrations of Mg2+ ions in a solution abolish Dps-nucleic acid interaction. The Mg2+ ion concentration should be adjusted based on the protein used. Another option to release nucleic acids from the complex is to disrupt the structure of the complex by precipitating the protein with ethanol or phenol, preferably phenol. Released polynucleotide can be resuspended in a low-salt solution, including water. The complex, or nucleic acid separated from the complex can be further purified. Purification methods will be readily known to a person skilled in the art. The available purification methods that can be applied according to the present disclosure are for example utilizing bi-phase system phenol:chloroform:isoamyl alcohol (e.g. in ratio 25:24:1), which denaturates the polynucleotide-binding protein while forces nucleic acid into water phase. Purification methods can include also re-precipitation of nucleic acids, chromatography, or using the advantage of ability of silica to bind nucleic acids in the form of nucleic acid-binding magnetic beads, where silica coated paramagnetic beads are added to the samples to bind polynucleotide (e.g. Agencourt AM Pure beads from Beckman Coulter), spin columns with silica-coated membrane (e.g. from QIAGEN) or any other silica-coated material. With regards to silica coated magnetic beads, the mixture of beads and nucleic acid are immobilized on magnets and washed to remove remaining protein and contaminants. Removal of residual binding solution is executed with a second wash solution and finally the polynucleotide is eluted in a low-salt buffer. Various suitable nucleic acid purification kits are commercially available. The obtained and optionally purified polynucleotide can be subjected to downstream applications, such as PCR and sequencing.

Use of the polynucleotide-binding protein, such as a Dps protein, in combination with known DNA/RNA purification methods offers further advantages. For example, use of the present solution in combination with known isolation methods allows to extract nucleic acids from much larger initial volumes, and to get higher amount of polynucleotides compared to extraction of the same polynucleotides solely by the presently known methods (such as separation by DNA-binding beads). Therefore, a polynucleotide-binding protein, such as a Dps protein, can be used to concentrate the initial sample.

The polynucleotide that is successfully obtained from a liquid biological sample, is separated from the complex and optionally purified, presents a diagnostic analyte that needs to be assayed for the presence of a biomarker. The term “assay” is used herein to refer to an act of identifying, screening, probing or determining by using any conventional means. For example, a biological sample or separated polynucleotide can be assayed for the presence of a specific biomarker by using polymerase chain reaction (PCR), sequencing, next generation sequencing, Northern blot analysis, Southern blot analysis, reverse transcription-polymerase chain reaction (RT-PCR), TaqMan-based assay, sequencing, high-density oligonucleotide SNP array, restriction fragment length polymorphism (RFLP) assay, dynamic allele-specific hybridization, primer extension assay, oligonucleotide ligase assay, analysis of single strand conformation polymorphism, temperature gradient gel electrophoresis (TGGE), denaturing high performance liquid chromatography, high resolution melting analysis, DNA mismatch-binding protein assay, capillary electrophoresis, immunoassay, ELISA, mass spectroscopy, or the like. Available technologies to interrogate nucleic acid are described also in Table 1 of Nature Reviews, April 2017, Vol 17, 223-38. In addition, the polynucleotide retrieved by the process as described herein can be assayed for epigenetic modifications, such as DNA methylation or other alterations that do not relate to changes in DNA sequence. To assay means herein to optionally subject a sample, for example the biological sample or the polynucleotide, to an analytical assay protocol, which may include for example purification, transformation or further preparation of the respective sample to bring it in a form suitable for analysis; and testing and/or measuring.

The term “biomarker” refers herein to a polynucleotide sequence or a copy number thereof, the presence or absence of which can be used to identify the existence of a disease, the state of the disease or the prognosis for disease development (predictive biomarkers and disease biomarkers, also condition biomarker) or to identify responders or non-responders to an active pharmaceutical ingredient or to predict or monitor toxicity, or for adjusting the dose of an active pharmaceutical ingredient with the aim to improve its safety and efficacy (therapeutic biomarker). For example, in oncology number of biomarkers are used to confirm the existence of a disease and often, at the same time to inform a decision about which active pharmaceutical ingredient needs to be administered to the subject. In the context of the present disclosure, the biomarker is a mutation, frame-shift mutation, functional mutation, deletion, translocation, insertion, duplication, or inversion of the DNA or RNA, alien DNA or RNA (for example a presence of viral DNA or RNA in a subject, or a presence or absence of fetal DNA) or DNA or RNA modification, or a combination thereof. DNA modification includes DNA alkylation or acylation, where for example cytosine or adenine DNA nucleotides are modified by addition of a methyl groups. mRNA modification includes RNA editing, which leads to a change of nucleotides after the sequence has been generated by RNA polymerase. Examples of such biomarkers in oncology are BRAF, KRAS, EGFR mutations, ALK or ROS translocations, UGT1A1, c-KIT translocation, CD30, FIP1L1-PDGFRalpha, PDGFR, PML/RAR-alpha, TPMT and Abl-bcr fusion. As an example of a possible biomarkers in the field of prenatal diagnosis differentially methylated sequences located at 21q22.3 (AIRE, SIM2 and ERG genes), 1q32.1 (CD48 gene and FAIM3 gene), 2p14 (ARHGAP25 gene) and 12q24 (SELPLG gene) were suggested as candidate biomarkers for non-invasive prenatal diagnosis of Down syndrome (Old et al. Reprod Biomed Online. 2007 August; 15(2):227-35). Biomarkers that relate to the disease, i.e. identify the disease characterized by a biomarker, were disclosed in biomedical literature or compiled in a database by a work as described in A. Bravo, M. Cases, N. Queralt, F. Sanz, L. I. Furlong, “A knowledge-driven approach to extract disease-related biomarkers from the literature”, BioMed Research International, volume 2014 (2014). The database of disease related-biomarkers can be accessed at http://ibi.imim.es/biomarkers/. In addition, the U.S. Food and Drug Administration (FDA) provides on its web pages a table of pharmacogenomic biomarkers in drug labelling. The table lists active pharmaceutical ingredients approved by the FDA with pharmacogenomic information found in the drug labelling. The presence of the biomarker in the drug label indicates specific actions to be taken based on the biomarker information. The methods and processes, kits and products disclosed herein can be applied to search for novel biomarkers.

The clinically relevant biomarkers, including new biomarkers, can be determined in a polynucleotide that is isolated from the liquid biological sample preferably by PCR or sequencing. PCR method can include multiplex PCR. Sequencing of captured nucleic acids (e.g. cfDNA) can be achieved by employing well known methods and product-kits in the art on Next-generation sequencing (NGD) and NGS-library preparation. The methods will generally entail several standard steps of NGS-library preparation, including end-repair and A-tailing, Adapter ligation and Library amplification, sequencing and the like. Sequencing can include next generation sequencing performed for example on any one of the next-generation sequencing platforms, such as 454 (Roche, including GS FLX(+) System), Illumina (Solexa) (including GA/HiSeq/MiSeq/NextSeq/MiniSeq/NovaSeq), SOLiD (Applied Biosystems, including 5500xl W Genetic Analyzer), Ion Torrent and PacBio (Pacific Biosciences). The information obtained during the analysis and assaying of a polynucleotide can then be interpreted in order to arrive at a meaningful conclusion about the relevancy of the biomarker for the clinical status of a subject. Based on the presence or absence of a biomarker a conclusion can possibly be drawn about the type, stage, or the like, of the disease, and possibly about treatment options, if any.

The sample can be any liquid biological sample. The sample can be selected from the group consisting of plasma, serum, blood, sputum, tear, urine, saliva, synovial fluid, pleural effusions and cerebrospinal fluid, or a lysate thereof. Preferably, the liquid biological sample is plasma, urine or serum, more preferably is serum. It will be immediately apparent to a skilled person that the sample will depend on the disease or a condition of the subject, or the information that is aimed to be obtained from the analysis. Equally, the selection of a sample will influence certain steps of a method of the present disclosure. For example, if the sample is blood, it may require first removal of the cells in the sample by filtration, centrifugation or other methods before the polynucleotide-binding protein is introduced to the sample. Alternatively, cells may need to be disrupted to release the nucleic acids and expose them to polynucleotide-binding protein. Further procedural steps that may be required for optimal use of the present disclosure are dilution of the sample, changing or adding a solvent, adjustment of the divalent metal ions, pH, conductivity or the like. It is expected that certain optimization will also depend on the protein used. However, the underlying principle of forming and precipitating a complex comprising a polynucleotide-binding protein and a polynucleotide from a sample is expected to remain the same. The polynucleotide-binding protein enables the extraction and analytical methods of liquid biopsy to be sufficiently robust and repetitive to have the method clinically validates and broadly used in clinical diagnostics. Whenever the biological sample, a polynucleotide-binding protein or a polynucleotide need to be diluted, resuspended, purified or the like with addition of water, a nuclease-free water (NFW) is preferably used. Such water is commercially available from various vendors such as ThermoFischer, Invitrogen and Ambion. Customarily, NFW has been deionized, filtered into the final bottle, and autoclaved.

One embodiment of the present disclosure is an isolated protein-polynucleotide complex comprising a polynucleotide-binding protein and a polynucleotide. “Isolated” according to the present disclosure means removed from the original biological sample. “Separated” as used herein means isolated or concentrated within a smaller volume. Depending on the separation method, the complex can be isolated already during the separation method, such as filtration, or chromatography. If, on the other hand, the separation method is centrifugation, the complex is isolated after decanting or removing the remainders of the biological sample from the formed pellet comprising the precipitate. In a preferred embodiment, the polynucleotide in the complex originates from a subject, preferably human. Human DNA is expected to be the relevant polynucleotide assayed in liquid biopsy in most cases. Preferably, the amount of the isolated protein-polynucleotide complex is such that contains enough polynucleotide for a meaningful analysis of a biomarker. In one embodiment, the isolated protein-polynucleotide complex comprises at least 3 ng, 12 ng, preferably at least 15 ng of the polynucleotides. In a preferred embodiment the isolated protein-polynucleotide complex is stable. The methods that include separation of a polynucleotide from the liquid biological sample can include isolation of said polynucleotide from the sample as part of the same method or can include an additional step to isolate the polynucleotide from the biological sample. The isolated protein-polynucleotide complex can be for example a stabilized biological material, which can be stored for later analysis, or shipped to a diagnostic laboratory for analysis. In addition, such isolated protein-polynucleotide complex can help form a repository of genetic material for later use, such as to form a bank of genetic material. For example, an isolated complex that contains cell-free DNA obtained from biological samples that were taken periodically over a period of time, can be stored to retain information of changes in nucleic acid material over time, e.g. from a tumor patient throughout their treatment or stages of disease diagnosis or progression, or from a person throughout his or hers life.

The principles described above can be efficiently employed in the use of a polynucleotide-binding protein in a method of comprising directly contacting a polynucleotide in a liquid biological sample from a subject in vitro with a polynucleotide-binding protein to form a complex which precipitates, in the use of the protein for the separation of the polynucleotide from the sample in the form of a complex, which in turn can be further purified, polynucleotide separated from the protein and assayed. Analogous embodiments of the present disclosure are represented by methods that apply the beneficial characteristics of the polynucleotide-binding protein. For example, the present invention discloses a method for protecting a polynucleotide in a biological sample comprising polynucleotides from a subject comprising a step of adding a polynucleotide-binding protein to the sample. The same method can be used to bind polynucleotides in a liquid biological sample from a subject. The strength of binding of the protein to the nucleic acid, i.e. forming a complex, can be adjusted as explained above, for example by adjusting the concentration of divalent cation ions in the sample. The binding can be also influenced by adjusting pH, solvent or the like.

Another embodiment of the present disclosure is a method for separation of polynucleotides from a liquid biological sample comprising polynucleotides from a subject, or an lysate thereof, the method comprising the steps of: (a) providing the sample, or the lysate thereof, comprising polynucleotides; (b) adding a polynucleotide-binding protein to the sample, or the lysate thereof, to form a protein-polynucleotide complex; and (b) separating the formed protein-polynucleotide complex from the sample, or the lysate thereof. Again, the separation of the precipitated complex can be effected by various means, for example chromatography, centrifugation, sedimentation, filtration, or utilizing magnetic properties of the polynucleotide-binding protein. The separated complex is thus ready for downstream handling as described above. The complex can be further purified, stored, the captured polynucleotide released from the complex, optionally purified again, and assayed to obtain information on a biomarker. The diagnostic results can then be assessed and translated into an appropriate diagnosis and/or treatment as described above.

The methods, steps, uses and the like disclosed above, can be applied as appropriately to the use of polynucleotide-binding protein in the in vitro diagnosis of a disease or a condition of a subject, wherein the polynucleotide-binding protein is used to separate a polynucleotide from a liquid biological sample from the subject comprising the polynucleotide; a method for detecting the presence of a biomarker in a biological sample from a subject, said method comprising:

(i) providing the liquid biological sample obtained from a subject comprising polynucleotides;

(ii) adding a polynucleotide-binding protein to the sample to form a protein-polynucleotide complex;

(iii) separating the formed protein-polynucleotide complex from the sample; and

(iv) assaying the polynucleotide to determine whether the biomarker is present in the liquid biological sample from said subject; a method comprising directly contacting a polynucleotide in a liquid biological sample from a subject in vitro with a polynucleotide-binding protein, thereby forming a protein-polynucleotide complex that precipitates in the sample; or a method of characterizing a disease or a condition of a subject comprising detecting the presence of a biomarker indicative of said disease or the condition in a liquid biological sample from the subject, the method comprising:

(i) providing the liquid biological sample obtained from a subject comprising polynucleotides;

(ii) adding a polynucleotide-binding protein to the sample to form a protein-polynucleotide complex;

(iii) separating the formed protein-polynucleotide complex from the sample; and

(iv) assaying the polynucleotide to determine whether the biomarker is present in the liquid biological sample from said subject.

A particular benefit of the present disclosure is that the volume of the biological sample is practically unlimited. The reason is that the polynucleotide-binding protein can be relatively easily prepared in large quantities, enabling sufficient reagent for treating large sample volumes. Once the protein has been added, the sample can be split into aliquots. Therefore, the present disclosure can be applied even on biological sample such as urine, (even cumulative 24-hour or 48-hour urine sample). By applying the same notion, larger volumes of e.g. subject's blood or plasma can be processed, or smaller volumes combined, to eventually yield sufficient quantities of polynucleotides that allow robust analysis. The present disclosure provides a powerful tool to substantially increase detection threshold and thus sensitivity of liquid biopsy methods.

The methods, kits, etc. disclosed herein can be applied to customary sample volumes in a laboratory, including sample volumes of between 1 μL and 2500 μL, for example about 1 μL, 10 μL, 50 μL, 100 μL, 1000 μL or 2000 μL. However, the most benefit of the robust, easy to produce and easy-to-scale-up methods described herein can be obtained when handling larger clinically relevant samples. In one embodiment, the volume of a liquid biological sample can be for example up to 1000 ml. In another embodiment, the sample volume is between 1.0 ml and 500 ml, preferably more than 2 ml and up to 1000 ml, preferably up to 500 ml. In yet another embodiment the sample has a volume of between 2.0 ml and 50 ml, 30 ml, or 7.5 ml, more preferably the sample has a volume of between 2.0 ml and 10.0 ml, between 2.7 ml and 10.0 ml, or between 2.7 ml and 6.0 ml. The method can include processing more than one aliquot and each aliquot has a volume of more than 2 ml, preferably at least 50 ml, particularly more than 5 ml and up to 1000 ml, up to 500 ml, or particularly more than 5 ml and up to 300 ml, or particularly more than 10 ml and up to 500 ml.

Herein disclosed is also a kit for use the methods according to the present disclosure comprising a polynucleotide-binding protein. The polynucleotide-binding protein can be presented in nuclease-free water, saline from said water, buffer, controlled release formulation, lyophilized powder, or other form compatible with other reagents, if any, of the kit. The kit can also comprise reagents to increase stability of the nucleic acid, its release from the complex or for its purification. Such reagent can be for example salt that dissociates in water at least into divalent cations, such as magnesium ions. The kit can also comprise a nuclease inhibitor, a protease or its inhibitor, pH modifier and optionally reagents for direct downstream application such as PCR or sequencing. The kit can also comprise a chelating agent. Such chelating agent can be for example EDTA. The reagents can be formulated separately or in combination, as powders or in a solution, such as buffer. The kit can be employed at sample collection or later to stabilize polynucleotide in a liquid biological sample. Alternatively, or in addition, it can be applied to facilitate separating the polynucleotide from a sample.

The advantages of a present disclosure can also be applied in preparation of a container for collection or storage of a biological sample from a subject comprising a polynucleotide-binding protein. As in a kit, the presence of the polynucleotide-binding protein in a container improves stability of the nucleic acids in the collected sample, as well as enables separation of the nucleic acids from larger volumes. Therefore, the container can easily accept a sample volume of at least 2.0 ml, or at least 4.0 ml. The container can be for example a tube, bottle or a bag, such as for example blood collection tubes or bags known in the art. The polynucleotide-binding protein can be added to or be combined with existing containers suitable for collecting or storing biological sample, such as EDTA blood collection tube (Vacutainer K2EDTA; Becton, Dickinson, Oxford, UK), or a Cell-Free DNA™ BCT (cfDNA BCT; Streck). The volume of the container can be of at least 2.7 ml, at least 4 ml, more than 10 ml, between 50 ml and 500 ml or at least 100 ml.

Both, the kit and the container can further contain inorganic and biochemical additives of different types. A suitable additive would be for example silica or sodium fluoride. Beside EDTA, trisodium citrate or a potassium oxalate can also be used. Trisodium citrate, which can be further buffered or not, is an anticoagulant. Another additive in the container or the kit can be Heparin (combined with a NA+, Li+ or NH4+ cation), which anticoagulates blood. Similar result can be obtained by adding a cocktail of citric acid, theophylline, adenosine and dipyridamole, which together hinder platelet activation after blood collection. When an opposite effect is required, i.e. to stimulate clotting, thrombin can be added. The kit and the container can also contain a thixotropic gel, which can be of various density. Said gel changes its viscosity based on the centrifugation force, enabling cells in a liquid biological sample to be separated from a supernatant by centrifugation. The kit can include reagents to prepare the obtained nucleic acids for creation of NGS-library or for next-generation sequencing. Such reagents can be end-repair reaction reagents like end-polishing buffer, end-polishing enzyme mix, nuclease free water, or adapter ligation reagents like ligation buffer, ligase and adapters. Therefore, in one embodiment the kit and/or the container disclosed herein comprises an additive or a reagent in addition to the polynucleotide-binding protein.

The data obtained by analysing nucleic acid (e.g. detecting a specific biomarker) that has been separated from a liquid biological sample from a subject can inform a decision, first on the condition or a disease of the subject. The information about the biomarker present can potentially give away the nature, stage, type, spread, severity of a disease or a condition, provided the biomarker has been validated for the respective clinical picture. Alternatively, the methods described herein can be used to build the link between a disease or a condition based on the new biomarkers found with the help of liquid biopsy. Once the disease or the condition have been identified and linked to a specific biomarker, analysing nucleic acid that has been isolated by the methods of the present disclosure can also inform about possible treatment option. Therefore, the present disclosure contemplates a method of selectively treating a subject having a disease that is sensitive to an active pharmaceutical ingredient and characterized by a biomarker, the method comprising a) selecting the subject for treatment with the active pharmaceutical ingredient on the basis of the subject having the biomarker, wherein the presence of the biomarker is determined on a polynucleotide that was separated from a liquid biological sample by polynucleotide-binding protein, and thereafter, administering a therapeutically effective amount of the active pharmaceutical ingredient to the subject.

By analogy, an active pharmaceutical ingredient can be used in a subject in the treatment of a disease characterized by a biomarker and being sensitive to the active pharmaceutical ingredient, the treatment comprising (i) assaying a sample obtained from the subject according to processes and methods as described above, (ii) determining if the subject has the biomarker, and (iii) if the biomarker is present, administering the active pharmaceutical ingredient to the subject. There have been many active pharmaceutical ingredient-biomarker pairs, or pharmaceutical ingredient-biomarker-disease triplets reported. The term “active pharmaceutical ingredient” as used herein refers to a chemical compound or a mixture thereof that is administered to a subject to treat a disease and elicits a therapeutic effect. The effect can be for example amelioration of a disease, reduction of symptoms of a disease, causing reduction in risk of progression of a disease, slowing development of a disease, slowing up progression of a disease, or causing a reduction in side effects of another active pharmaceutical ingredient etc. The terms “treat”, “treating” or “treatment” as used herein in connection to a disease or a disorder refer to ameliorating a disease or disorder. It can also refer to alleviating or ameliorating at least one physical parameter that is indicative of a disease. A “therapeutically effective amount” refers herein to an amount effective, at dosages and for periods of time necessary, to achieve the desired therapeutic result. Examples of the active pharmaceutical ingredients which benefit from the biomarker analysis of a biological sample from a subject are vemurafenib (BRAF V600E mutation), crizotinib, ASP3026, CH5424802, or AP26113 (ALK or ROS1 translocation), trastuzumab, pertuzumab, ado-trastuzumab emtansine (HER-2/neu gene amplification), trametinib, dabrafenib (BRAF V600E and V600K mutation, single nucleotide variants), erlotinib, afatinib, gefitinib (exon 19 deletions and exon 21 (L858R) substitution mutations of the epidermal growth factor receptor (EGFR) gene, G719X, exon 20 insertions, T790M, 57681 and L861Q), osimertinib (T790M EGFR mutation, G719X, exon 20 insertions, T790M, 57681 and L861Q), afatinib, necitumumab, nimotuzumab, PF299804, R05083945, ABT-806, or AP26113 (EGFR mutations), pembrolizumab (PD-L1 expression), cetuximab, panitumumab (seven somatic mutations in codons 12 and 13 of the KRAS gene), olaparib (mutations, deletions insertions and duplications in BRCA1 and BRCA2 genes), enasidenib (single nucleotide variants (SNVs) coding nine IDH2 mutations (R140Q, R140L, R140G, R140W, R172K, R172M, R172G, R172S, and R172W), panitumumab (56 specific mutations in RAS genes [KRAS (exons 2, 3, and 4) and NRAS (exons 2, 3, and 4), midostaurin (FLT3 Mutation), and venetoclax (deletion).

Since the present disclosure provides means to render liquid biopsy techniques more robust and easier to validate, the use of a polynucleotide-binding protein in methods of in vitro diagnostics, or ex vivo diagnostics, is expected to cut clinical development times for a new active pharmaceutical ingredient. By replacing solid biopsies and an ability to monitor one or more biomarkers at more time points during a clinical study could shorten clinical trials and help gather more relevant information faster. Therefore, a method of developing an active pharmaceutical ingredient is contemplated herein, comprising contacting a polynucleotide-binding protein with a liquid biological sample from a subject having a disease and thereafter assaying said sample for the differential level of a biomarker compared to a standard level of the biomarker, wherein the standard level of the biomarker is the level (i) before the treatment with the active pharmaceutical ingredient, (ii) of a control, or (iii) at a different time point; and the differential level of the biomarker is indicative of treatment of the disease with the active pharmaceutical ingredient. The control can be a level of the biomarker of a healthy subject, generally set value, the level in a subject from a comparator arm of the clinical study, or the like.

Production and Purification of a Polynucleotide-Binding Protein Production of a protein can be achieved by employing well known methods in the art on how to clone, express and purify a recombinant protein. The methods will generally entail preparing cDNA, selection of a suitable vector, host organism and the like. In relation to vector further variants are possible in relation to a promotor, affinity marker, selection tags and the like. An organism of choice can be Escherichia coli. Production of a protein from E. coli has been extensively discussed by German L. Rosano and Eduardo A. Ceccarel in Front Microbiol. 2014; 5: 172. Further source of information on protein production and purification methods can be obtained in Nat Methods. 2008 February; 5(2): 135-146. Exemplary methods relating to Dps, including those of preparing Dps mutant strains and Dps variants can be found in Journal Bacteriology, 2013, doi: 10.1128/JB.00059-13; Journal Bacteriology, October 2015, Vol 197, 19, 3206-15 and Journal of Biological Chemistry, 1999, Vol 274, 46, 33105-13. Those documents contain also other general teaching on Dps proteins.

Applications of the Disclosure

Because the method of the present invention is useful with both single and double stranded NAs, as well as a wide range of nucleic acid fragment sizes, it is applicable in essentially any context in which nucleic acid, preservation, accumulation and/or separation from any biological liquid or liquified sample containing polynucleotides is desired. The methods of the present disclosure can be applied in oncology (for example to help detect biomarkers as described by J. Twomey et al. in Drug Resistance Updates 30 (2017) 48-62), transplant medicine (for example as described by Dany Anglicheau et al. in Transplantation 2016; 100: 2024-2038), in the context of infectious diseases, caused either by bacteria or virus, immunology, including autoimmune disease settings, cardiovascular diseases or the like. Cardiovascular disease includes, without limitation, diseases such as diabetes mellitus, hypertension, ischemic heart disease, rheumatic heart disease, or inflammatory heart disease. The present disclosure can be applied in detection of a biomarker (such as genetic biomarkers) described in Journal of Lipids Volume 2015, Article ID 971453, 50 pages by Ravi Kant Upadhyay; BioMed Research International, Volume 2017, Article ID 9158572, 6 pages, by Laurent Metzinger, Stefano de Franciscis, and Raffaele Serra; or in Coronary artery disease-associated genetic variants and biomarkers of inflammation by Morten Krogh Christiansen, published on Jul. 7, 2017. In addition, the present disclosure can be beneficially applied in paediatrics. An example of non-invasive method where the present invention can be utilized is analysis of urine from children. Such analysis can be advantageous when presence of a virus or specific bacteria would need to be determined. The present disclosure can be beneficially put into practice also in forensic analysis or genetic anthropology, where DNA from bones or teeth is isolated and stability of it needs to be preserved during isolation and handling.

TABLE 1

| | |
|---|---|
| Amino acid | |
| sequence | |
| SEQ ID NO: 1 | mstaklyksk atnllytrnd vsdsekkatv |
| | ellnrqviqf idlslitkqa hwnmrganfi |
| | avhemldgfr talidhldtm aeravqlggy |
| | algttqvins ktplksypld |
| | ihnvqdhlkeladryaivan dvrkaigeak |
| | dddtadilta asrdldkflw fiesnie |
| Nucleotide | |
| sequence | |
| SEQ ID NO: 2 | atgagtaccg ctaaattagt taaatcaaaa |
| | gcgaccaatc tgctttatac |
| | ccgcaacgatgtctccgaca gcgagaaaaa |
| | agcaacagta gagttgctga atcgccaggt |
| | tatccagtttattgatcttt ctttgattac |
| | caaacaagcg cactggaaca tgcgcggcgc |
| | taacttcattgccgtacatg |
| | aaatgctgga tggcttccgc accgcactga |
| | tcgatcatct ggataccatggcagaacgtg |
| | cagtgcagct gggcggtgta gctctgggga |
| | ccactcaagt tatcaacagcaaaaccccgc |
| | tgaaaagtta cccgctggac atccacaacg |
| | ttcaggatca cctgaaagaactggctgacc |
| | gttacgcaat cgtcgctaat gacgtacgca |
| | aagcgattgg cgaagcgaaagatgacgaca |
| | ccgcagatat cctgaccgcc gcgtctcgcg |
| | acctggataa attcctgtggtttatcgagt |
| | ctaacatcga ataa |

Here is disclosed amino acid and nucleotide sequence information of an exemplary polynucleotide-binding protein, such as Dps protein NP_415333.1.
The table includes an amino acid sequence of the protein known under the accession number NP_415333.1, indicated under SEQ ID NO: 1, and the nucleotide sequence of the corresponding genome DNA gene encoding the protein in Escherichia Coli str. K-12 substr. MG1655, indicated as SEQ ID NO: 2.
The protein served as an example of a suitable polynucleotide-binding protein that can be applied according to the present disclosure.
Based on the present disclosure it would be in a purview of a skilled person to select other suitable polynucleotide-binding proteins, or if needed, to test their suitability for the uses disclosed herein based on their ability to form a precipitating complex with a polynucleotide in water as described above.

### EXAMPLES

Preparation of a Dps Protein

Dps protein was expressed and purified according to Karas et al. (Karas V O, Westerlaken I, Meyer A S. 2013. Application of an in vitro DNA protection assay to visualize stress mediation properties of the Dps protein. J Vis Exp 75:50390). Briefly, protease-deficient strain of E. coli (such as BL21(DE3) pLysS) was transformed with a pET vector (such as pET17) into which the Dps protein-encoding gene sequence has been cloned. Cells were grown at 37° C. with shaking at 250 rpm to an optical density at 600 nm of 0.4 to 0.6, and expression of Dps was induced by addition of isopropyl-D-thiogalactopyranoside (IPTG) to a concentration of 0.3 mM, and incubated at 37° C. for 3-4 hr while shaking. Cells were harvested by centrifugation at 6,000×g for 15 min and pellet resuspended in 7.5 ml of DEAE buffer A (50 mM HEPES-KOH, pH 7.5, 100 mM NaCl, 0.1 mM EDTA) per L of induced cell culture. To prevent degradation of over expressed Dps, a mixture of protease inhibitors was added. Cells were lysed using French press and clarified of insoluble particles by centrifugation at 30,000×g for 35 min at 4° C. after clarification, the lysate was purified with a combination of ion-exchange column chromatography (e.g. DEAE-Sepharose as AEX and e.g. SP Sepharose as CEX chromatography media) and ammonium sulfate precipitation. The concentration of purified Dps protein (NP_415333.1) samples was determined by measuring the absorbance at 280 nm, with a molar extinction coefficient of 15,470 M⁻¹ cm⁻¹ for the Dps monomer. Dps concentration in the final storage buffer (50 mM HEPES-KOH, pH 7.5, 50 mM NaCl) was ˜7 mg/mL. The following experiments were performed by using the prepared Dps protein as a representative polynucleotide-binding protein.

### Example 1. Separation of DNA from Solution Using Dps Protein

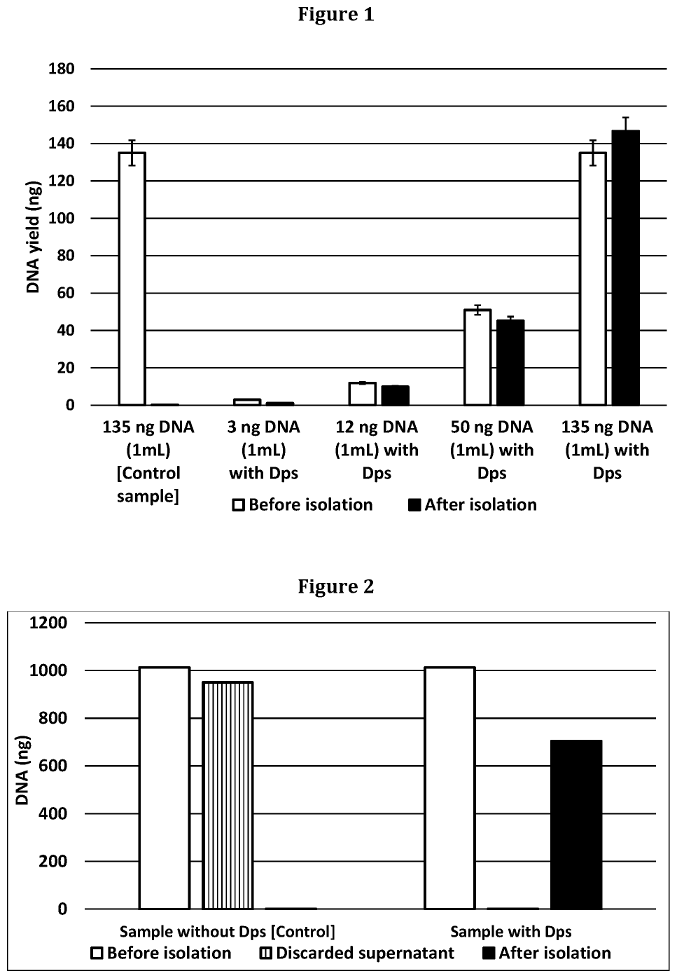

To demonstrate the functionality of Dps protein for separating nucleic acids from a solution, a proof-of-concept experiment was performed. To this end, 1 mL solutions with various DNA concentrations were prepared by suspending DNA standard (NEB, 100 bp ladder) in nuclease-free water (NFW; Nuclease-free Water (Ambion®)). Additionally, 10 μL of the prepared Dps was added and samples were incubated at 33° C. for 30 min. After incubation, samples were centrifuged at 21000×g for 15 min at 4° C. and supernatant was discarded. Pelleted Dps-DNA complex (i.e. biocrystals) was resuspended in 50 μL water. Proteinase K was added and samples were incubated at 56° C. for 10 min in order to release Dps from DNA. Subsequently, DNA was purified with DNA-binding magnetic beads (i.e. 90 μL magnetic beads were used, following two consecutive washes with 80% ethanol). DNA was eluted from magnetic beads with 50 μL water and DNA concentration was measured. All DNA measurements were performed fluorometrically. The isolation process using Dps protein resulted in more than 90% recovery of initial DNA amount in all Dps containing samples (FIG. 1). Recovery was not dependent on the initial DNA concentration in the sample. On the other hand, DNA solution without added Dps protein (control sample) resulted in the complete loss of DNA. In summary, this experiment confirms that DNA can be separated from solution using Dps.

### Example 2. Scalability of Biofluid Sample Volumes Using Dps Protein

The functionality of Dps protein to enable accumulation of DNA from larger-volume solutions is assessed. DNA was suspended in 100 mL water and the prepared solution was then split to obtain two 50 mL DNA samples. The prepared Dps protein (100 μL) was added to only one DNA sample. Both samples—with and without Dps—were incubated at room temperature (RT) for 45 min, followed by 30 min centrifugation step (4000 rpm at 4° C.). Supernatant was discarded by decanting and Dps-DNA containing pellet was resuspended in 1 mL water. The isolation principle was similar as in the example 1, however, after pellet resuspension, DNA-Dps complex (biocrystals) were not further treated with proteinase K and/or purified using DNA-binding magnetic beads. No DNA was detected in discarded supernatant from sample with added Dps, while at least 94% of initial DNA was lost through discarded supernatant in sample without Dps (FIG. 2). DNA isolation process using Dps protein resulted in recovery of more than 68% of initial DNA amount, compared to the DNA sample without Dps, where DNA was not detected in the final eluate (FIG. 2). Note that concentration of DNA in a sample processed with Dps may be underestimated due to remainder of the active Dps-DNA interaction. In addition, higher amounts of Dps or even surplus of Dps is expected to lead to higher yields. In conclusion, DNA can be successfully collected and concentrated from larger volume solutions using Dps.

### Example 3. RNA Separation from Solution Using Dps

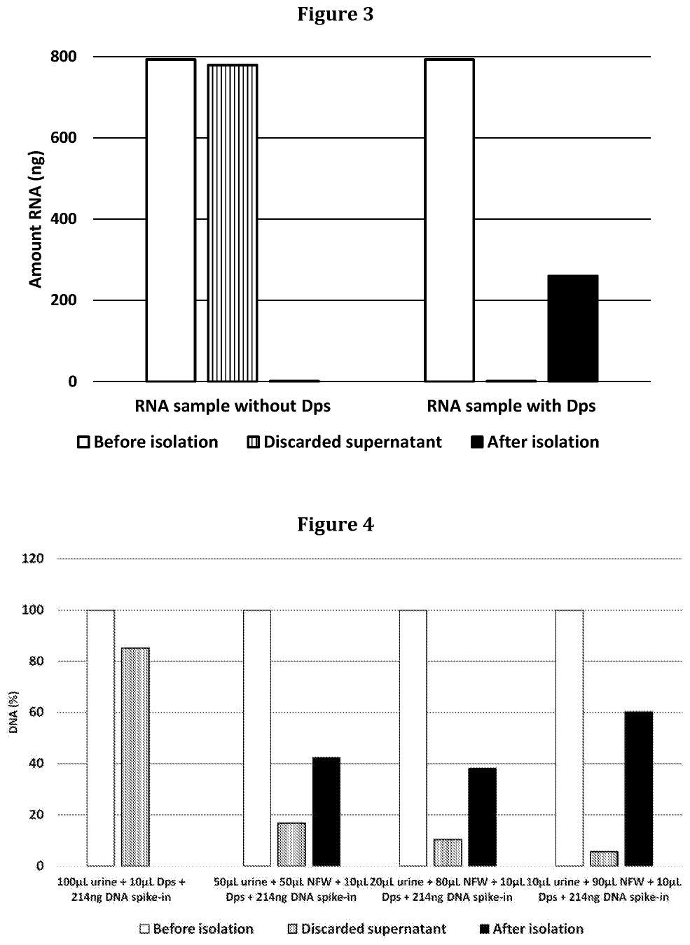

Dps protein was shown before to bind RNA. To further demonstrate that the capacity of Dps protein to bind RNA allows separation of RNA-protein complex from a solution, a proof-of-concept experiment was performed. First, RNA solutions were prepared by suspending about 800 ng RNA in 1 mL of water. RNA solutions were prepared with 10 μL of the prepared Dps protein and without adding the Dps protein, respectively. After 10-minute incubation at 4° C. and subsequent centrifugation (21000×g, 10 min at 4° C.) supernatant was discarded and pellet containing Dps-RNA complex (biocrystals) was resuspended in 50 μL water. RNA amount was measured fluorometrically in: (1) the starting solution, (2) discarded supernatant after centrifugation and (3) in final 50 μL eluate. Results show, that while RNA in a solution without Dps was lost through discarded supernatant, roughly 33% of initial RNA from Dps-containing solution was recovered in final eluate (FIG. 3). As in Example 2 low recovery rate may be the result of the undermeasurement of RNA due to still active Dps-RNA interaction and high protein (i.e. Dps) content in small-volume solution. On the other hand, optimization of experimental parameters (including Dps amount, additional RNA purification, etc.) is expected to improve RNA recovery. Overall, we have confirmed that RNA can be separated from RNA-containing solution using a Dps protein. In addition, it was observed that incubation of a sample at lower temperatures such as 4° C. facilitates formation of the complex between a Dps protein and polynucleotides and increases the amount of recovered polynucleotides from the sample.

### Example 4. Extraction of DNA from a Liquid Biological Sample (Biofluid) Using Dps Protein

To demonstrate the functionality of Dps protein for separating nucleic acids from a liquid biological sample (biofluid), such as urine, a proof-of-concept experiment was performed. Urine sample from a healthy individual was collected and immediately centrifuged at 3000×g for 10 min at 4° C. to obtain cell free solution. Biofluids contain divalent cations, including Mg2+ at high concentrations. Therefore, in order to obtain suitable concentration of divalent cations for Dps protein to bind polynucleotide efficiently, urine sample was diluted with nuclease-free water (NFW; Nuclease-free Water (Ambion®) at dilution factors of 1 (no dilution), 2 (50 μL urine in 50 μL NFW), 5 (204 urine in 80 μL NFW) and 10 (10 μL urine in 90 μL NFW), respectively. DNA (i.e. 222 bp long PCR amplicon at concentration 214 ng DNA/4 μL NFW) was spiked-in and Dps (104) were added to 100 μL of each prepared dilution. Samples were incubated for 30 min at RT, followed by centrifugation step (20.000×g/15 min/4° C.). Supernatant was discarded and pellet was resuspended in 114 μL NFW. DNA concentrations were measured fluorometrically in: (1) the starting solution, (2) discarded supernatant after centrifugation and (3) in final 114 μL eluate. Results are shown in FIG. 4. Dps was not functional in non-diluted sample, where over 80% of DNA was lost through discarded supernatant. In contrast, DNA was successfully collected in all diluted samples. Dilution 1:10 resulted in best DNA recovery (over 60%). In conclusion, these results support applicability of Dps in separating nucleic acids from liquid biological samples (biofluid), such as urine, once suitable adjustments (e.g. concentration of divalent cations) are made that promote formation of the complex (biocrystals). It is expected that process optimization as to the concentration of divalent cations, pH, amount of the polynucleotide-binding protein added to the biological sample or the like, can further increase the yield.

### Example 5. Extraction of cfDNA from Larger Volume of Urine Sample Using Dps

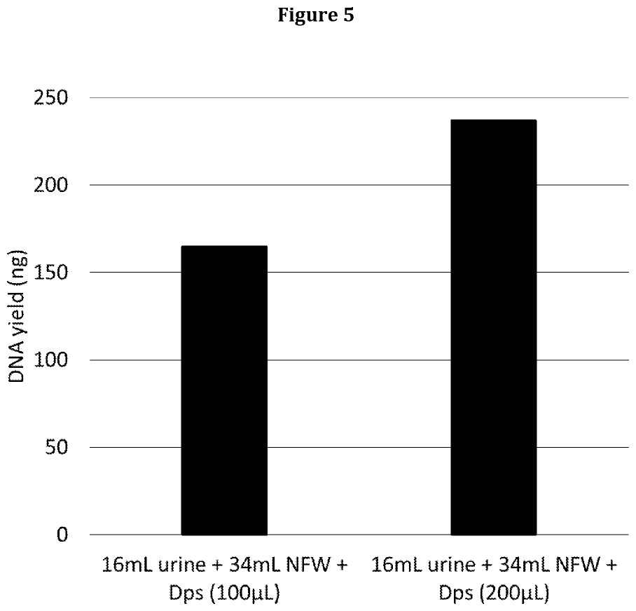

To demonstrate applicability of Dps for accumulating cfDNA from larger volumes of liquid biological sample (biofluid), a proof-of-concept experiment was performed. Urine sample from a healthy individual was collected and immediately centrifuged at 3000×g for 10 min at 4° C. to obtain cell free solution. Solution was split into two centrifuge tubes (16 mL each) and diluted with nuclease-free water (NFW) to a total volume of 50 mL in each tube. Different volumes of Dps, 100 μL and 200 μL, respectively, were added and prepared solutions were left shaking at slow speed for 90 min at RT. Following incubation, samples were centrifuged at 16000×g for 15 min at 4° C. Supernatant was discarded and pellet was resuspended in 400 μL water. Final DNA concentration was measured fluorometrically. Results are shown in FIG. 5. Solution with added 100 μL of Dps resulted in 165 ng of collected DNA (i.e. 10 ng/mL of urine), while 237 ng of DNA were obtained (i.e. 15 ng/mL of urine) from a solution where 200 μL was added. Results show that cfDNA can be successfully extracted from larger urine volumes using Dps. Moreover, extracted DNA yield is positively correlated with the amount of added Dps (FIG. 5). In conclusion, results prove the ability to extract and accumulate DNA from large-volume urine sample by using Dps.

### Example 6. Extraction of cfDNA from Plasma Using Dps

To demonstrate applicability of Dps for extraction of cfDNA from biofluid, such as plasma, a proof-of-concept experiment was performed. Blood sample from one healthy individual was collected in BD Vacutainer® K2E (EDTA) Tubes, kept below RT and processed within one hour. Sample was centrifuged at 3000×g for 10 min at 4° C. After centrifugation, plasma sample was transferred into fresh centrifuge tube and centrifuged again at 16000×g for 10 min at 4° C. to remove residual blood and cell debris. After centrifugation, 8 mL of cell-free plasma was diluted with 32 mL of water (membrane-filtered and autoclaved). Dps (280 μL) was added and solution was shaken at slow speed for 120 min at RT. Following incubation, sample was centrifuged at 16000×g for 15 min at 4° C. Supernatant was discarded and pellet was resuspended in 400 μL water. Final DNA concentration was measured fluorometrically. Overall, 134 ng of DNA was obtained, resulting in 16.8 ng of cfDNA per 1 mL of plasma. Resuspended pellet was further purified using the QIAamp DNA Blood Mini Kit (Qiagen) following manufacturer guidelines and purified DNA was analysed on capillary electrophoresis on chip using 2100 Bioanalyzer Instrument (Agilent Genomics) in order to determine the size of the captured DNA. A clear and distinct peak with average size of 176 bp (spanning 150-200 bp) was observed. In summary, cfDNA was collected successfully and in high amounts from larger volumes of plasma using Dps.

### Example 7. Sequencing of Nucleic Acids

Cell free DNA accumulated and separated from large volume biofluid samples such as urine or blood (e.g. as per Example 4 and 5) can be further processed with appropriate NGS-library preparation kits to obtain samples ready for next-generation sequencing. NGS-library preparation generally entails several standard steps, including end-repair and A-tailing, adapter ligation and library amplification. For all NGS-library preparation kits, purified (using of example silica coated paramagnetic beads or spin columns with silica-coated beads) cell-free DNA could be used as input. Alternatively, Dps-DNA complex treated with proteinase (such as proteinase A) could be used as input. Moreover, since most initial end-repair reaction mixes contain free Mg2+, which can disrupt the DNA-Dps complex (might not be applicable for all NGS-library preparation kits), the best alternative also resulting in highest yields would be the direct use of Dps-DNA complex as the input DNA.

Described here is library preparation from captured cfDNA using QIAseq Ultralow Input library lit (QIAGEN).

As input DNA, 10 pg-100 ng of purified cell-free DNA can we used. Alternatively, DNA-Dps complex could be used directly as DNA input, since most enzymes in the initial end-repair reaction require free Mg2+, which activate disruption of DNA-Dps complex (might not be applicable for all NGS library preparation kits). End-repair reaction mix is set up on ice by mixing input DNA, End-polishing Buffer (5 μL), End-polishing Enzyme Mix (2 μL) and nuclease free water (up to 50 μL total volume) and incubated at 25° C. for 30 min followed by incubation at 65° C. for 15 min. After incubation, the reaction mix is put on ice and components for adapter ligation, including Ligation buffer (25 μL), ligase (5 μL), QIAseq Adapter (2 μL) and nuclease-free water (18 μL) are added. Reaction mix is further incubated at 25° C. for 10 min. Following adapter ligation and library amplification steps, reaction cleanup and removal of residual adapter dimers is achieved by using Agencourt AM Pure XP beads. Finally, PCR-based library amplification using the QIAseq HiFi PCR Master Mix that is included in the kit is required, if the input cfDNA amount is below 10 ng. Prepared sample is ready for sequencing using next-generation technologies.

### Example 8. Shielding DNA from DNase I Cleavage

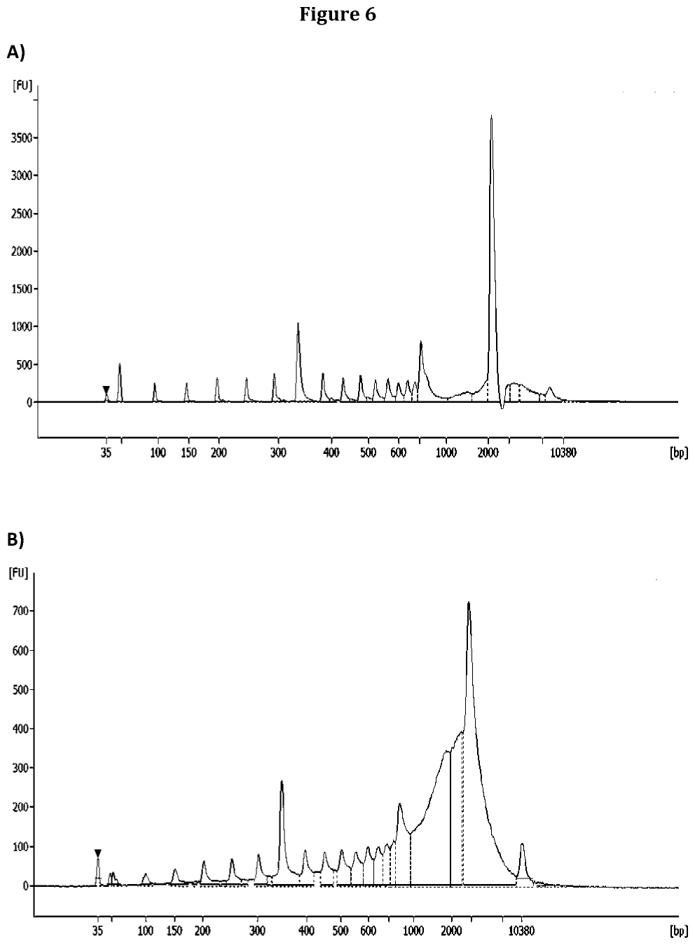

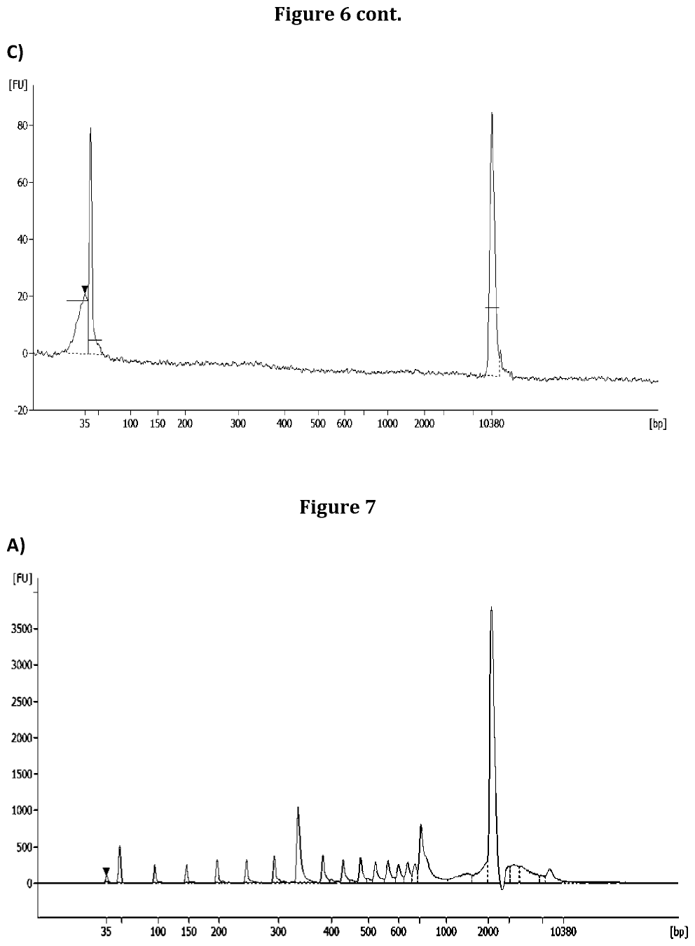

To demonstrate the functionality of Dps protein to protect DNA against DNase cleavage, a proof-of-concept experiment was performed. To this end, two separate DNA spike-in solutions were prepared, where DNA standard (50 bp DNA Ladder, ThermoFische Scientific) was suspended in nuclease-free water (NFW; Nuclease-free Water [Ambion®]), followed by the addition of the Dps (30 μL) to only one solution. Both samples were then incubated for 30 min at RT. After incubation, DNase I (8 Kunitz units; QIAGEN) was added to both samples resulting in two final 300 μL solutions. After a 9-day storage at RT, DNA was extracted from 100 μL of each sample using QIAamp DNA Blood Mini Kit (Qiagen) and analysed using Bioanalyzer 2100 (Agilent Technologies). Results show, that Dps was able to significantly protect DNA from DNase cleavage since ladder-specific sizes of the input DNA (especially low molecular weight sizes below 400 bp) were clearly observed. On the other hand, no DNA was observed in the Dps-free sample, suggesting its complete degradation by DNase cleavage activity (FIG. 6).

### Example 9. Shielding DNA from Degradation in Urine Sample

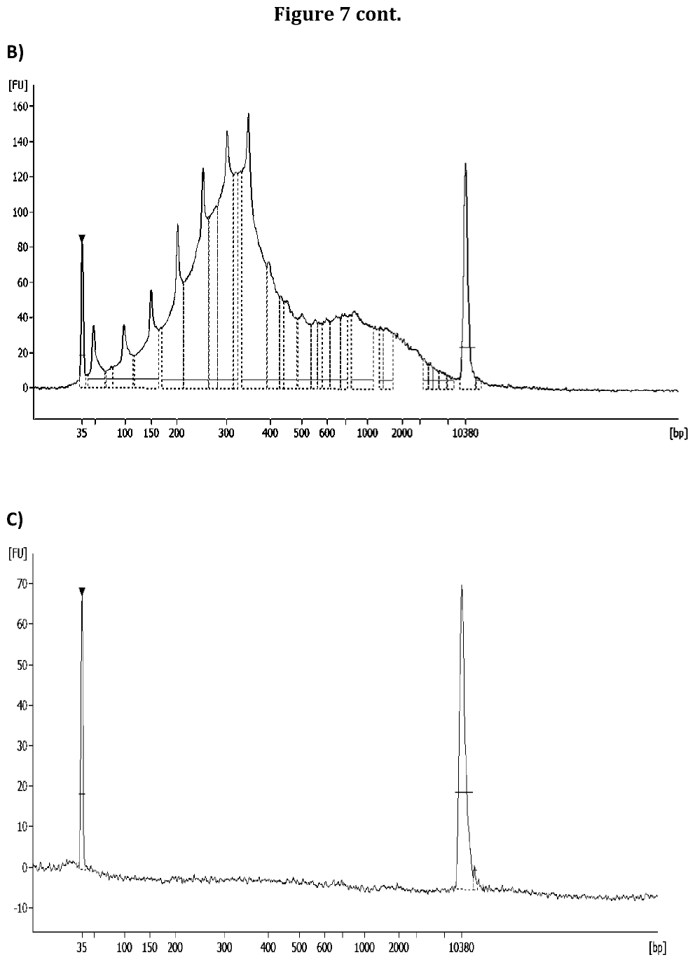

To demonstrate applicability of Dps protein to stabilize DNA in a liquid biological sample (biofluid), a proof-of-concept experiment was performed. Urine sample from a healthy individual was collected and immediately centrifuged at 16000×g for 10 min at 4° C. to obtain cell free solution. Solution was split into two microcentrifuge tubes (90 μL each) and diluted with spiked-in DNA-containing (50 bp DNA Ladder, ThermoFische Scientific) nuclease-free water (NFW) to a final volume of 270 μL in each tube. Additionally, 30 μL of Dps protein was added to one tube, while 30 μL of NFW was added to the other tube, instead. Samples were stored at RT for 9 days. After 9 days, DNA was extracted from 100 μL of each sample using QIAamp DNA Blood Mini Kit (Qiagen) and analysed using Bioanalyzer 2100 (Agilent Technologies). FIG. 7 suggests that Dps protein was able to successfully protect low molecular weight DNA (sizes below 400 bp) from degradation, as distinct-sized fragments of the spiked-in DNA are clearly visible. However, results suggest that higher molecular weight DNA fragments (from gDNA contamination), were not effectively protected by Dps protein. DNA in a Dps-free sample had been degraded below the level of detection, since no DNA was observed.

### Example 10. Accumulation of cfDNA from a Larger Volume of Plasma and Subsequent Purification Using QIAGEN Kit

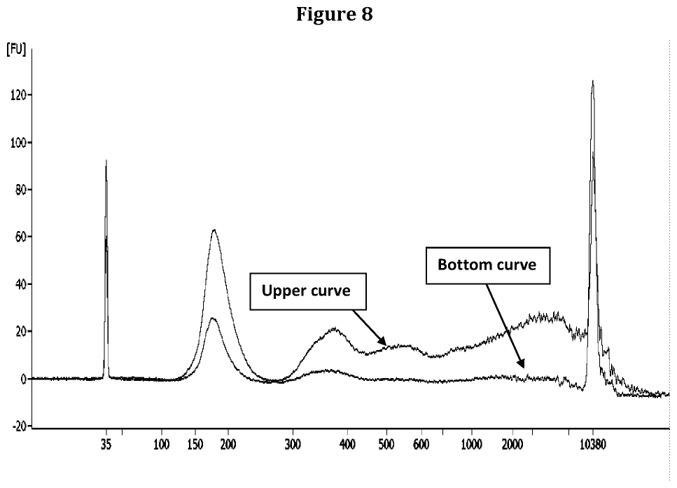

A proof-of-concept experiment was carried out to demonstrate that the cfDNA accumulated from a large volume of biological sample with Dps is compatible with existing and commercially available DNA isolation kits for further purification. To this end, cfDNA was extracted from two cell free plasma samples from a single healthy donor using solely the QIAamp MinElute ccfDNA Mini Kit (QIAGEN) and a combination of the QIAamp MinElute ccfDNA Mini Kit after Dps had been added to the sample, respectively. Using solely the QIAamp MinElute ccfDNA Mini Kit, cfDNA was extracted from 1 mL of cell-free plasma following manufacturer's instructions. The extraction of cfDNA with Dps in combination with QIAamp MinElute ccfDNA Mini kit was performed as follows. First, 7.5 mL of cell-free plasma was diluted in 30 ml of nuclease-free water and 3004 of the Dps was added. After incubation by gentle mixing at RT for 80 minutes, the solution was centrifuged at 16.000×g for 15 min at 4° C. Supernatant was carefully decanted and pellet was resuspended in 1000 μL nuclease-free water. Obtained Dps-cfDNA solution was further purified with QIAamp MinElute ccfDNA Mini kit as per manufacturer's instructions. Then, the DNA from both extractions was visualized using Bioanalyzer 2100 (Agilent Technologies). Results show that applying Dps and extracting DNA with the help of Dps and with commercially available cfDNA purification kit (in this case QIAamp MinElute ccfDNA Mini kit by QIAGEN) allows extraction of higher-yields of cfDNA from larger sample volumes using the equal amount of reagents provided in the commercial kit (FIG. 8). It should be noted that the QIAamp MinElute ccfDNA Mini kit (without the use of Dps) allows extraction of cfDNA from only up to 4 mL of plasma sample. This results clearly show that a Dps protein can easily be added to or combined with available extraction or purification methods. A Dps protein allows, for example, concentrating polynucleotides from larger volumes before they are further purified by other known processes, or stabilizes them before being processed.

1. A method comprising directly contacting in vitro a polynucleotide in a liquid biological sample from a subject in vitro with a polynucleotide-binding protein, thereby forming a protein-polynucleotide complex that precipitates in the sample.

2. The method according to claim 1, wherein the polynucleotide-binding protein is used to separate a polynucleotide from a liquid biological sample from a subject comprising the polynucleotide.

3. The method according to claim 1, wherein the polynucleotide-binding protein is used to protect a polynucleotide in the biological sample comprising the polynucleotide.

4. (canceled)

5. (canceled)

6. (canceled)

7. The method according to claim 1, wherein the protein-polynucleotide complex is separated from the biological sample by chromatography, centrifugation, sedimentation or filtration of the sample, or by utilizing magnetic properties of the polynucleotide-binding protein.

8. (canceled)

9. The method according to claim 1, wherein the polynucleotide is further released from the protein-polynucleotide complex.

10. (canceled)

11. (canceled)

12. (canceled)

13. (canceled)

14. (canceled)

15. (canceled)

16. (canceled)

17. The method according to claim 1, wherein the polynucleotide is further assayed.

18. (canceled)

19. The method according to claim 1, wherein the polynucleotide is further assayed by sequencing.

20. (canceled)

21. The method according to claim 1, wherein the results of the assay are interpreted.

22. The method according to claim 1, wherein the liquid biological sample is plasma, serum, blood, sputum, tear, urine, saliva, synovial fluid, pleural effusions, cerebrospinal fluid, or a lysate thereof, or a cell or a tissue lysate.

23. (canceled)

24. (canceled)

25. (canceled)

26. (canceled)

27. The method according to claim 1, wherein the liquid biological sample has a volume of more than 0.05 ml and up to 500 ml.

28. (canceled)

29. (canceled)

30. (canceled)

31. (canceled)

32. (canceled)

33. The method according to claim 1, wherein the polynucleotide-binding protein is a prokaryotic protein, wherein the protein is a heatshock protein, ribosome protein, MCVV protein, ferritin, apoferritin, a Dps protein, a Dps-like protein, or a mixture thereof.

34. The method according to claim 1, wherein the polynucleotide-binding protein is a Dps protein or a Dps-like protein.

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. A kit comprising a polynucleotide-binding protein for use in a method comprising directly contacting in vitro a polynucleotide in a liquid biological sample from a subject in vitro with the polynucleotide-binding protein, thereby forming a protein-polynucleotide complex that precipitates in the sample.

72. The kit according to claim 71 further comprising reagents for release or purification of the polynucleotide.

73. (canceled)

74. (canceled)

75. (canceled)

76. (canceled)

77. The kit according to claim 71 for stabilizing polynucleotide in the sample.

78. (canceled)

79. (canceled)

80. The kit according to claim 71 for separating the polynucleotide from a liquid biological sample comprising polynucleotide from a subject.

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. (canceled)

94. (canceled)

95. (canceled)

96. (canceled)

97. (canceled)

98. (canceled)

99. (canceled)

100. (canceled)

101. (canceled)

102. (canceled)

103. (canceled)

104. (canceled)

105. (canceled)

106. (canceled)

107. (canceled)

108. (canceled)

109. (canceled)

110. A method of characterizing a disease or a condition of a subject comprising detecting the presence of a biomarker indicative of said disease or the condition in a liquid biological sample from the subject, the method comprising:
(i) providing the liquid biological sample obtained from a subject comprising polynucleotides;
(ii) adding a polynucleotide-binding protein to the sample to form a protein-polynucleotide complex;
(iii) separating the formed protein-polynucleotide complex from the sample; and
(iv) assaying the polynucleotide to determine whether the biomarker is present in the liquid biological sample from said subject.

111. (canceled)

112. The method according to claim 110, wherein the polynucleotide is cell-free nucleic acid (cfNA).

113. (canceled)

114. The method according to claim 110 for use in oncology, transplant medicine, infectious disease, immunology, autoimmune disease, or cardiovascular disease.

115. (canceled)

116. (canceled)

117. The method according to claim 110, wherein the polynucleotide-binding protein is a polynucleoide-binding protein from a strain of Streptococci, Listeria, Helicobacter or Escherichia.

118. (canceled)

119. (canceled)

120. (canceled)

121. (canceled)


SEQUENCE LISTING (2 sequences)

SEQ ID NO: 1 - protein, 167 aa, Escherichia coli [sequence omitted]

SEQ ID NO: 2 - DNA, 504 bp, Escherichia coli
atgagtaccgctaaattagttaaatcaaaagcgaccaatctgctttatacccgcaacgatgtctccgacagcgagaaaaaagcaacagtagagttgctgaatcgccaggttatccagtttattgatctttctttgattaccaaacaagcgcactggaacatgcgcggcgctaacttcattgccgtacatgaaatgctggatggcttccgcaccgcactgatcgatcatctggataccatggcagaacgtgcagtgcagctgggcggtgtagctctggggaccactcaagttatcaacagcaaaaccccgctgaaaagttacccgctggacatccacaacgttcaggatcacctgaaagaactggctgaccgttacgcaatcgtcgctaatgacgtacgcaaagcgattggcgaagcgaaagatgacgacaccgcagatatcctgaccgccgcgtctcgcgacctggataaattcctgtggtttatcgagtctaacatcgaataa